(19) United States
(12) Patent Application Publication
(10) Pub. No.: US 2012/0232642 A1
(43) Pub. Date: Sep. 13, 2012

(54) Punching-based stent and its manufacturing method

(75) Inventors: Chia Nan Wang, Kaohsiung City (TW); Wei Ming Hsiung, Kaohsiung City (TW)

(73) Assignee: Chia Nan Wang, Kaohsiung City (TW)

(21) Appl. No.: 13/137,526

(22) Filed: Aug. 24, 2011

(30) Foreign Application Priority Data
Mar. 7, 2011 (TW) 100107586
Apr. 29, 2011 (TW) 100115108

(51) Int. Cl. A61F 2/82; B23P 17/04

(52) U.S. Cl. 623/1.15; 29/592

(57) ABSTRACT

The present invention is to provide a stent comprising a main body or at least a strut and a principal manufacturing method by means of one punching die for precision punching machining without a follow-up rolling & precision welding step in order to fulfill improved yield rate, mass production, effectively curtailed throughput time and manufacturing cost, and stent's lowered price benefiting more patients.

### BACKGROUND OF THE INVENTION

1) Field of the Invention

The present invention relates to a stent and a method of manufacturing the stent, especially to a stent and a method of manufacturing the stent without a welding or rolling process in order to fulfill improved yield rate, mass production, effectively curtailed throughput time and manufacturing cost, and stent's lowered price.

2) Description of the Prior Art

Among 10 causes of deaths in developed nations, cardiovascular diseases always rank top three. As one blood vessel around the heart, the coronary artery provides oxygen and nutrition necessary to systole and diastole but is developed to be stenosis, arteriosclerosis or occlusion which causes some symptoms of anoxia such as angina pectoris and asthma, namely coronary artery disease, in case of fat (or cholesterol) accumulated on walls of the coronary artery and resulting in plaque.

The coronary artery with its diameter narrowed more than 60%˜70% of the normal diameter will lead to insufficient blood supplied to the heart. Furthermore, acute vascular occlusion followed by angina pectoris will further induce acute myocardial infarction and serious myocardial injury.

The diagnosis most effective in coronary heart diseases is so-called “cardiac catheterization” in addition to pharmaceuticals controlling symptoms. In this regard, a surgeon usually executes Percutaneous Transluminal Coronary Angioplasty (PTCA) via skin of one patient suffering from a coronary heart disease.

Additionally, referring to FIGS. 1a and 1b which indicate a stent used to be implanted into an appropriate patient's coronary artery during PTCA under a surgeon's recommendations for dilatation of the artery and no more stenosis.

During PTCA, a stent implanted into a patient's narrowed blood vessel for its diameter dilated is effective in blood flow substantially increased and symptoms of a coronary heart disease significantly restrained. After 1˜3 months, the stent will be covered by grown vascular endothelial cells but not exposed to a blood.

There are three methods used in manufacturing stents usually. The first one is to use the high-power laser machining technology in which a stainless steel pipe is cut to reticular-structure stents with unnecessary parts melted and removed by laser. This machining technology is hard to be operated because of sophisticated 3D machining and positioning during cutting which may result in some drawbacks such as deteriorated material strength due to high temperature laser, blood flow (blood cells or other ingredients in blood) probably obstructed (destroyed) by burrs or rough surfaces which are remained on edges of a finished stent, expensive equipment for laser machining, and complex patterns difficultly fabricated.

The second method for stent manufacturing is to use electro-discharge machining with some advantages shown as follows: different electrode designs for multiple machined holes with controllable depths (sizes) and precise positions completed in an NC or a CNC program which is very convenient in use; however, some disadvantages of electro-discharge machining are slow speed, 2D plane machining only, and electrode difficultly manufactured in the case of excessively complicated slots.

The third method for stent manufacturing is to use photolithography, i.e., photo resist coated on a metal pipe and developed by masks for removal of metal unprotected by photo resist with metal immersed into an etchant solution; some disadvantages of this method are: no one-step forming 3D cylindrical reticular stent, i.e., a stent manufactured with planar photolithography and etching processes followed by rolling, rolling and welding steps; a reticular-structure stent difficultly controlled or failing due to probable anisotropic or irregular etching with metallic etching solution sideway intruding any mask-shielded portion.

Accordingly, it is necessary to find a method of manufacturing stents without disadvantages in the prior arts for improved yield rate, mass production, effectively curtailed throughput time and manufacturing cost, and lowered price for more stent users.

### SUMMARY OF THE INVENTION

To solve the said problems, the present invention is to provide a punching-based stent and a method to manufacture a stent comprising a main body or at least a strut; the method to manufacture a stent is to employ a punching die for precision punching machining in which there is no precision welding process for the present invention after a rolling & forming step.

Accordingly, the principal object of the present invention is to provide a punching-based stent and a method of manufacturing the stent in order to fulfill improved yield rate, mass production, effectively curtailed throughput time and manufacturing cost, and lowered price benefiting more patients

To this end, the present invention has the principal technical measures delivered with the following techniques. The present invention is a punching-based stent used to support a narrowed or occlusive blood vessel and comprising a stent with a main body and a plurality of bulges: the bulges designed in the main body's two opposite lateral margins and arranged in staggered patterns; the main body comprising a plurality of struts and at least a supporting strut; the bulges comprising at least an auxiliary strut; the supporting strut installed in the main body's at least one lateral margin and connected to the struts as well as the bulges' auxiliary strut.

The purposes and the technical issues with respect to the present invention are further embodied with the following technical measures.

In the said punching-based stent, the struts are developed to be patterns intersecting mutually or not intersecting mutually.

In the said punching-based stent, the auxiliary strut is developed to be cannular-arranged patterns intersecting mutually or not intersecting mutually.

In the said punching-based stent, the strut comprises at least a punched hole or at least an indentation; or the auxiliary strut comprises at least a punched hole or at least an indentation.

In the said punching-based stent, the strut (or the auxiliary strut) has a thickness between 0.01 mm and 0.3 mm.

In the said punching-based stent, the stent is manufactured in (composite) metallic materials such as stainless steel, cobalt alloy, nickel, tantalum, and titanium-nickel alloy or polymer materials.

An additional technical measure adopted in the present invention is: A punching-based stent used to support a narrowed or occlusive blood vessel and comprising a stent with not less than one strut bended to at least two supporting struts in which there is at least one fold developed at theses supporting struts' intersection.

The purposes and the technical issues with respect to the present invention are further embodied with the following technical measures.

In the said punching-based stent, an included angle developed by the supporting struts should be less than 180 degrees.

In the said punching-based stent, the strut comprises at least a punched hole or at least an indentation.

In the said punching-based stent, the stent is manufactured in (composite) metallic materials such as stainless steel, cobalt alloy, nickel, tantalum, and titanium-nickel alloy or polymer materials.

A further technical measure adopted in the present invention is: A punching-based stent used to support a narrowed or occlusive blood vessel and having a stent with a main body which comprises a plurality of struts and at least a supporting strut: the struts arranged to be patterns not overlapping one another; the supporting strut installed in the main body's at least one lateral margin and connected to the struts.

The purposes and the technical issues with respect to the present invention are further embodied with the following technical measures.

In the said punching-based stent, the strut comprises at least a punched hole or at least an indentation.

In the said punching-based stent, the strut's thickness is kept between 0.01 mm and 0.3 mm.

A method of manufacturing a punching-based stent described in the present invention has steps shown as follows: a. Select one material used to manufacture a stent; b. Design and draw a stent's patterns; c. Manufacture a punching die according to the stent's patterns designed and drawn; d. Develop at least one stent Work-in-Process (WIP) from the material by means of the punching die for precision punching machining; e. Complete the stent with burrs and rough surfaces on the stent removed.

The purposes and the technical issues with respect to the present invention are further embodied with the following technical measures.

In the said method of manufacturing a punching-based stent, the patterns drawn in Step b comprise a main body and a plurality of bulges.

In the said method of manufacturing a punching-based stent, the patterns drawn in Step b comprise at least a strut bended to not less than two supporting struts in which there is one fold at the supporting struts' intersection.

In the said method of manufacturing a punching-based stent, there is at least one jig used to clamp or position the material for a precision punching machining process successfully performed by a punching die during Step d.

In the said method of manufacturing a punching-based stent, there is one post-inspection process adopted to check integrity of a stent manufactured in Step d.

In the said method of manufacturing a punching-based stent, there is one rolling & forming process after Step d by which the stent WIP could be rolled to be a cylindrical shape;

In the said method of manufacturing a punching-based stent, some substances such as heparin and titanium oxide or sprayed degradable (or non-degradable) polymer film agents can be applied to or coated on the stent's surface.

In contrast to prior arts, the present invention is to provide a stent comprising a main body or at least a strut in a manufacturing method for a precision punching machining process by a punching die without a precision welding process. As a result, the manufacturing method features improved yield rate, mass production, curtailed throughput time and effectively reduced manufacturing cost, and lowered price benefiting more patients intending to use stents.

### BRIEF DESCRIPTIONS OF THE DRAWINGS

FIG. 1a illustrates a stent, which is manufactured in the prior arts, implanted into a narrowed blood vessel but not dilated.

FIG. 1b illustrates a stent, which is manufactured in the prior arts, implanted into a narrowed blood vessel and dilated.

FIG. 2 is the first front view with respect to the first embodiment of the present invention.

FIG. 3 is the second front view with respect to the first embodiment of the present invention.

FIG. 4a illustrates a cannular auxiliary strut in the first embodiment of the present invention.

FIG. 4b illustrates a reticular auxiliary strut in the first embodiment of the present invention.

FIG. 4c illustrates an undulated auxiliary strut in the first embodiment of the present invention.

FIG. 5 is the side view with respect to the first embodiment of the present invention.

FIG. 6 is the cross-sectional view with respect to the first embodiment of the present invention.

FIG. 7 is the front view with respect to the second embodiment of the present invention.

FIG. 8 is the side view with respect to the second embodiment of the present invention.

FIG. 9 is the cross-sectional view with respect to the second embodiment of the present invention.

FIG. 10 is the flow diagram of manufacturing the present invention for the first and second embodiments.

FIG. 11 is the front view of the first type of stent in the third embodiment of the present invention.

FIG. 12 is the front view of the second type of stent in the third embodiment of the present invention.

FIG. 13 is the front view of the third type of stent in the third embodiment of the present invention.

FIG. 14 is the front view of the fourth type of stent in the third embodiment of the present invention.

FIG. 15 is the flow diagram of manufacturing the present invention in the third embodiment.

FIG. 16 is the side view with respect to the third embodiment of the present invention.

FIG. 17 is the cross-sectional view with respect to the third embodiment of the present invention.

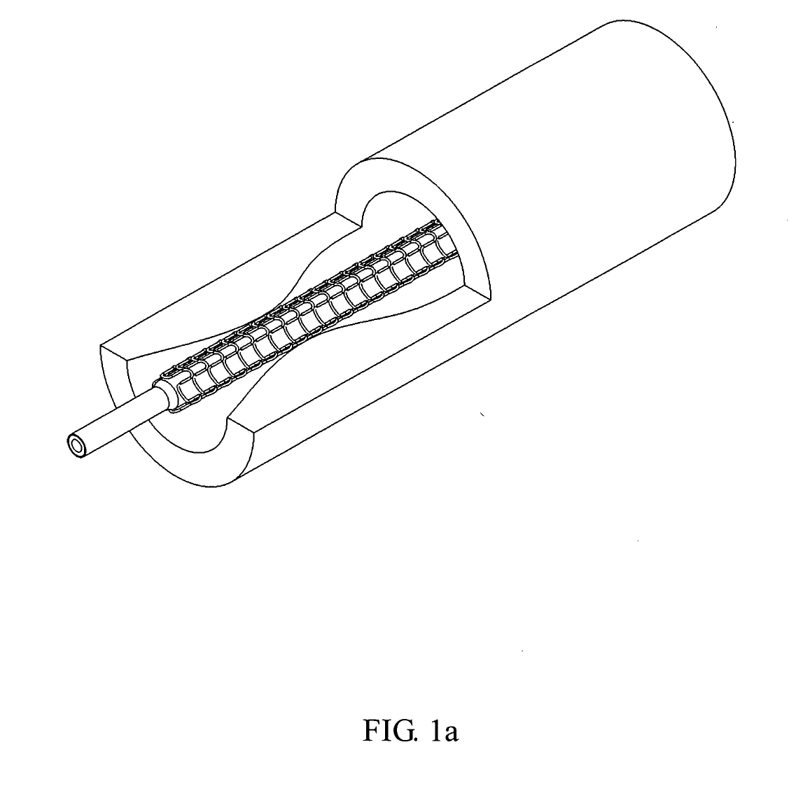

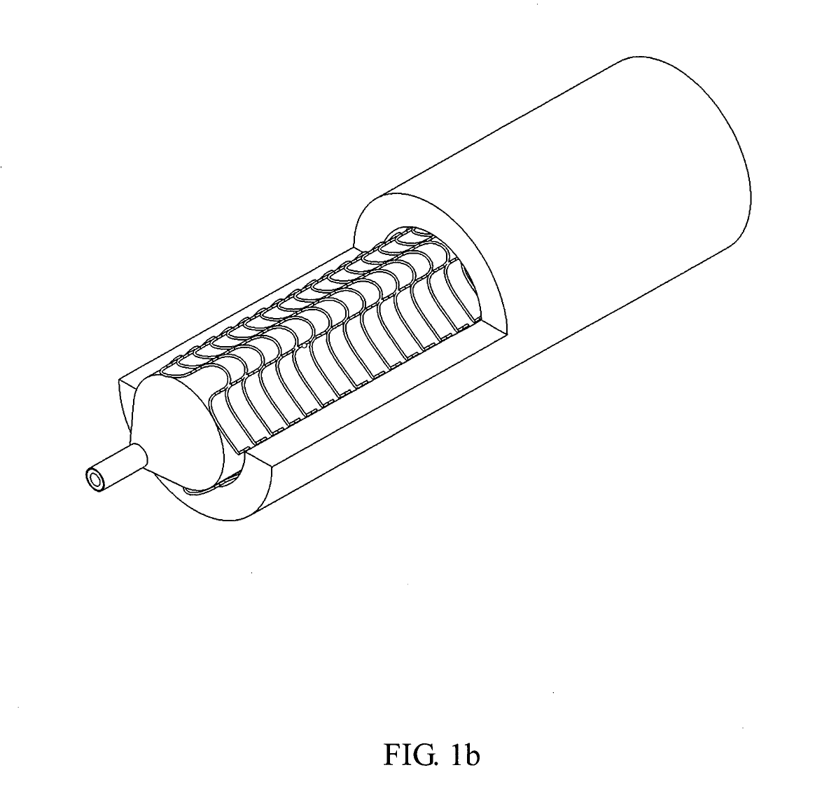

### DETAILED DESCRIPTIONS OF THE PREFERRED EMBODIMENTS

For objects, characteristics, and effects obviously and easily understood, the preferred embodiments of the present invention are particularly interpreted as follows:

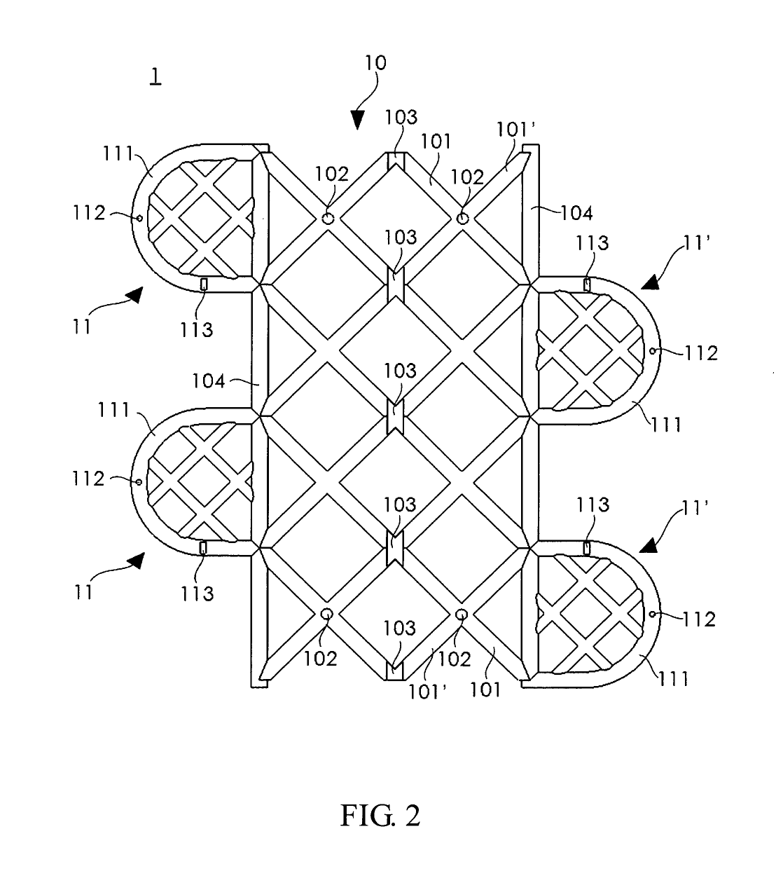

As shown in FIGS. 2˜6 which illustrate the first embodiment of the present invention of a punching-based stent used to support a narrowed or occlusive blood vessel, the present invention comprises a stent (1) with a main body (10) and a plurality of bulges (11) wherein the bulges (11) are developed in two opposite lateral margins of the main body (10) and arranged in staggered patterns.

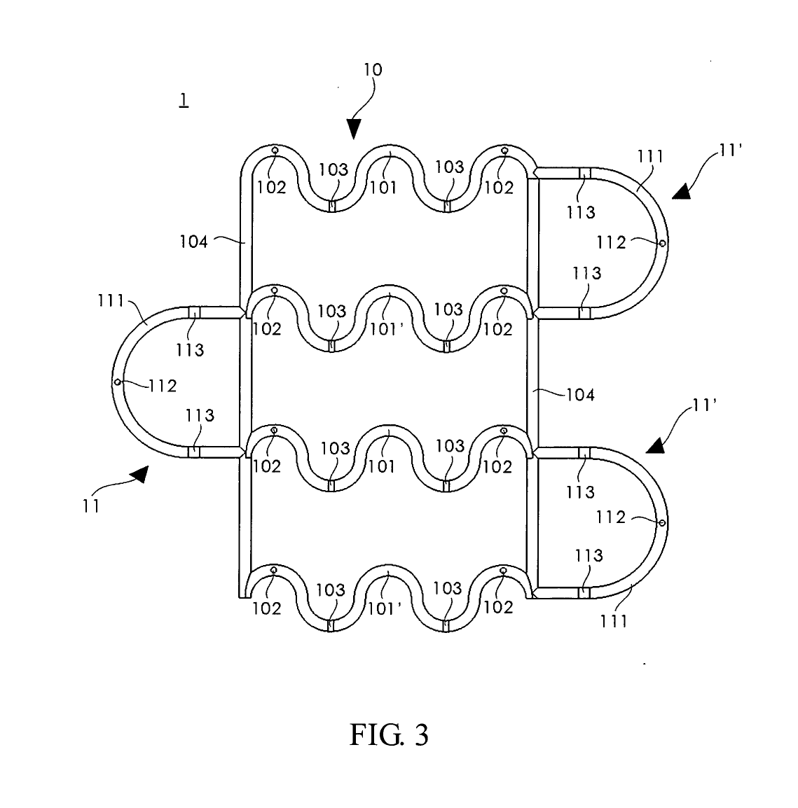

Specifically, the main body (10) comprises a plurality of struts (101) and at least a supporting strut (104) wherein the struts (101, 101′) could be distributed in either staggered patterns such as a reticular structure (FIG. 2) or non-staggered patterns such as an undulated structure (FIG. 3). The struts' structure in the present invention, however, should not be limited to staggered or non-staggered patterns which have been referred to as common knowledge in this field.

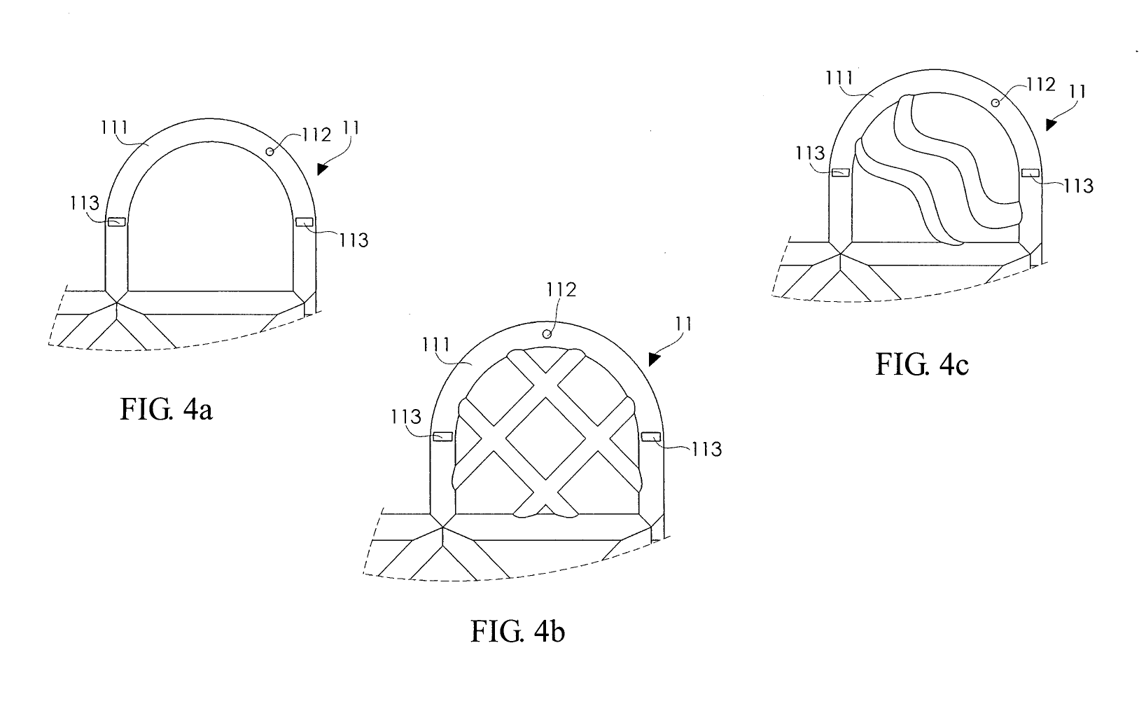

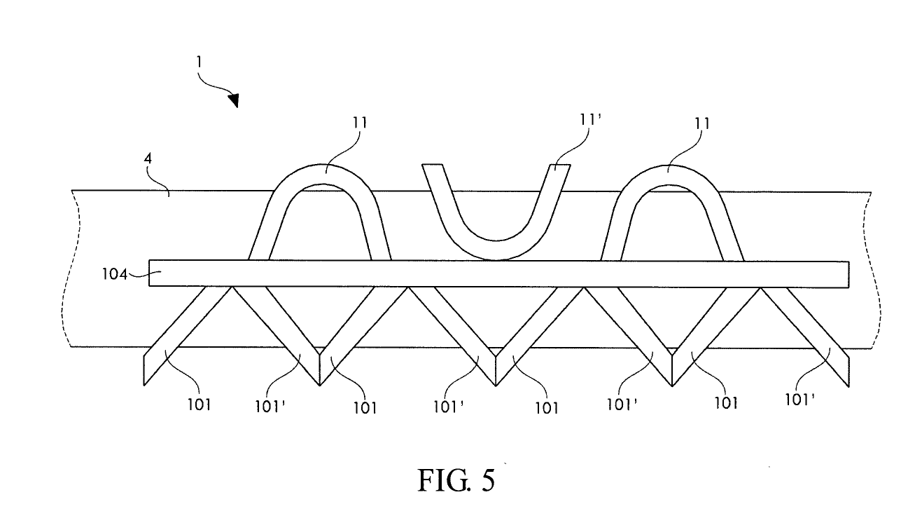

Specifically, the bulge (11) comprises at least an auxiliary strut (111), e.g., a cannular structure as shown in FIG. 4a, or a plurality of auxiliary struts (111) developed to be either staggered patterns, e.g., a reticular structure as shown in FIG. 4b, or non-staggered patterns, e.g., an undulated structure as shown in FIG. 4c. Similarly, the auxiliary strut's structure in the present invention, however, should not be limited to staggered or non-staggered patterns which have been referred to as common knowledge in this field.

In detail, the supporting strut (104) installed in at least one lateral margin of the main body (10) and coupled with both struts (101) and an auxiliary strut (111) of bulges (11) allows the main body (10) and the bulges (11) to become a one-piece structure design.

Preferably, the strut (101) also comprises at least a punched hole (102) or at least an indentation (103). Preferably, the auxiliary strut (111) also comprises at least a punched hole (112) or at least an indentation (113). A punched hole or an indentation contributes to any sprayed curative medicine adhered to a stent's surface.

In detail, the thickness (101T) of the strut (101) or the thickness (111T) of the auxiliary strut (111) is kept between 0.01 mm and 0.3 mm.

Preferably, the stent (1) is manufactured in (composite) metallic materials such as stainless steel, cobalt alloy, nickel, tantalum, and titanium-nickel alloy or polymer materials.

Referring to FIGS. 7˜9 which illustrate the second embodiment of the present invention of a punching-based stent used in supporting a narrowed or occlusive blood vessel wherein symbols interpreting the same structural characteristics as shown in the first embodiment and FIGS. 2˜6 are also indicated in FIGS. 7˜9 or neglected and not repeatedly described hereunder. Different from that of the first embodiment, the stent (1) of the second embodiment comprises at least a strut (12) bended to not less than two supporting struts (121a, 121b) in which at least one fold (13) is developed at their intersection.

Specifically, an included angle (A) developed by supporting struts (121a, 121b) is less than 180 degrees.

Preferably, the strut (12) also comprises at least a punched hole (102) or at least an indentation (103) which contributes to any sprayed curative medicine adhered to a stent's surface. Preferably, the thickness (12T) of the strut (12) is kept between 0.01 mm and 0.3 mm.

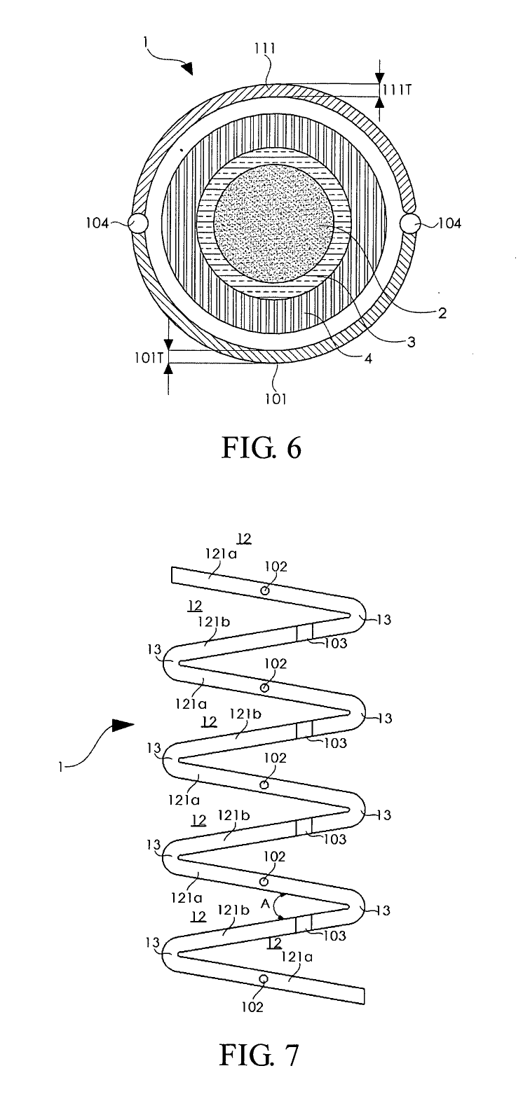

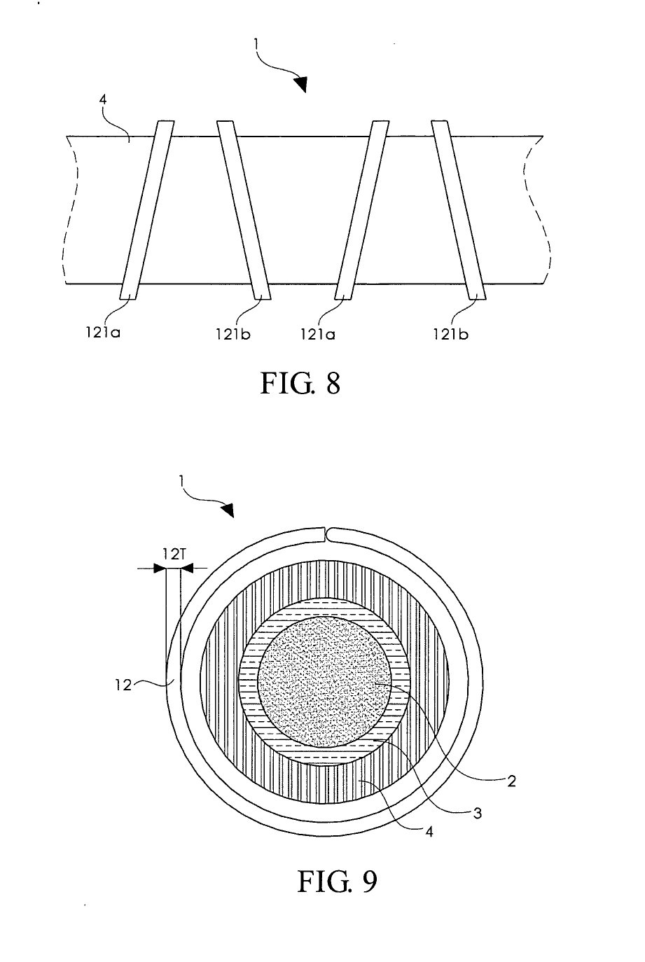

Referring to FIGS. 6 and 9 which illustrate an axle (2) comprising a cardiac catheterization (3) and a non-dilated balloon (4), which will dilate and expand the stent up to a required diameter during PTCA, on one external surface and penetrating the stent (1) during practice of the present invention actually. Depending on any application and a section of one blood vessel for a cardiac catheterization to be implanted, those factors such as a stent's length, thickness, shape and dimension, an axle's dimension and a stent's diameter with a balloon dilating during PTCA should not confine the present invention. As one prior art, PTCA with any stent implanted into a narrowed or occlusive blood vessel is not further described herein.

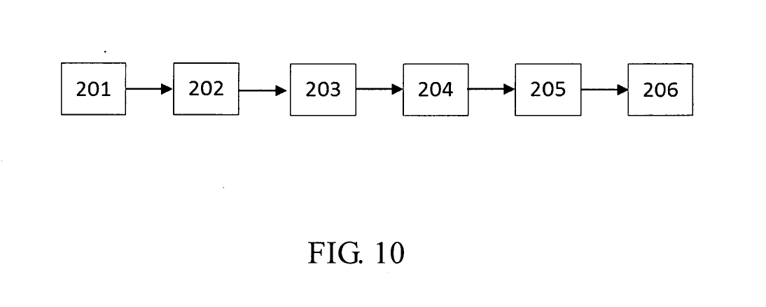

Referring to FIG. 10 which illustrates the first and second embodiments of the present invention of a punching-based stent delivered with steps as follows:
a. Select one material used to manufacture a stent (201);
b. Design and draw a stent's patterns (202);
c. Manufacture a punching die according to the stent's patterns designed and drawn (203);
d. Develop at least one stent Work-in-Process (WIP) from the material by means of the punching die for precision punching machining (204); and
e. Complete the stent with burrs and rough surfaces on the stent removed (206).

Preferably, the patterns drawn in Step b comprise a main body and a plurality of bulges which are located at the main body's two opposite lateral margins and arranged in staggered patterns;

Or, the patterns drawn in Step b comprise at least a strut bended to not less than two supporting struts in which there is at least one fold at these supporting struts' intersection.

Specifically, the stent's patterns designed and drawn in Steps b & c as well as manufactured for one punching die can be any patterns completed in Computer Aided Design (CAD) or the software Pro/engineering.

In addition, the size and the strength of a stent before and after expansion since Step b can be analyzed in a package for finite element analysis. The structural strength of a punching die since Step c is also analyzed in a package for finite element analysis.

Preferably, the main body and bulges mentioned in Step b comprise at least a punched hole or at least an indentation which contributes to any sprayed curative medicine adhered to a stent's surface. In this regard, the pharmaceutical substances including but not limited to heparin, titanium oxide or sprayed degradable (or non-degradable) polymer film agents can be applied to or coated on the stent's surface after Step e.

Preferably, a programmable precision punching machine along with at least one jig clamping and positioning the material for the process of precision punching machining successfully performed are used to execute Step d.

Furthermore, the thickness of the strut or the auxiliary strut of the stent developed in Step d is kept between 0.01 mm and 0.3 mm.

Preferably, there could be one post-inspection procedure to check integrity of a stent's structure manufactured in Step d, for instance, accuracy of the stent's appearance or a strut's size inspected in a Scanning Electron Microscope (SEM) to make sure of no irregular line width, break, burr, or rough surface in a stent's structure.

Preferably, there could be one rolling & forming step following Step d in order to roll the stent WIP to become a cylindrical shape (205) by means of a mechanical jig. In the first embodiment, it is unnecessary to transfer the present invention with correspondingly staggered bulges into a precision welding process after a rolling & forming step. Additionally, the bulges on the cylindrical stent could be developed to be overlapping or non-overlapping styles but depends on any required application or a blood vessel in which a stent will be implanted, so that the present invention is not restricted by the bulges.

Specifically, a stent's burrs or rough surfaces could be removed during a procedure for electrolysis & polish by an electro-chemical polishing machine in Step e; the stent's surface is also cleaned in a procedure for mirror polish which smoothes the stent's edges.

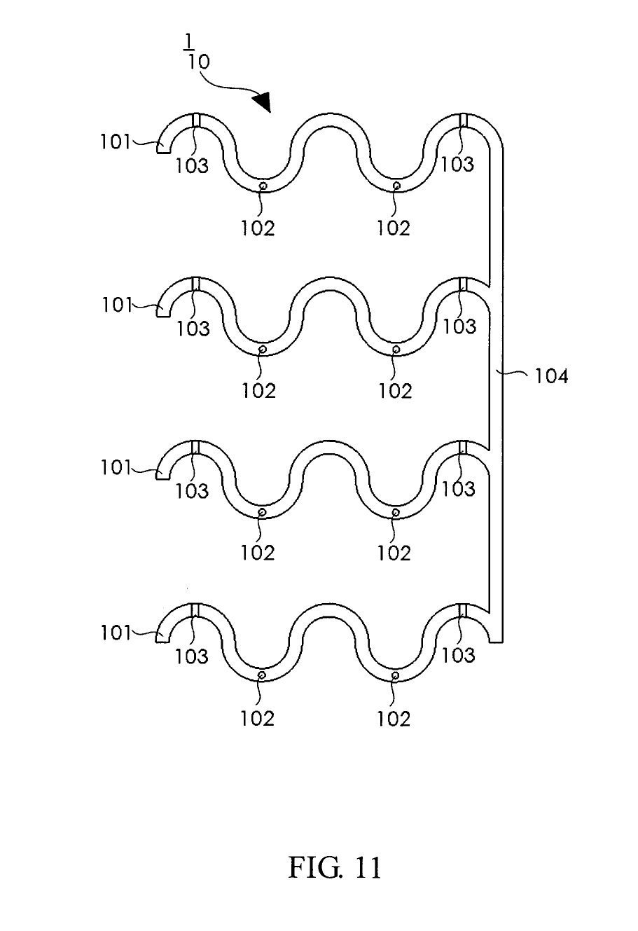

Referring to FIGS. 11˜17 which illustrate the third embodiment of the present invention of a punching-based stent used to support a narrowed or occlusive blood vessel wherein symbols interpreting the same structural characteristics as shown in the first embodiment and FIGS. 2˜6 are also indicated in FIGS. 11˜17 or neglected and not repeatedly described hereunder.

Different from that of the first embodiment, the stent (1) of the third embodiment comprises the main body (10) only without any bulge.

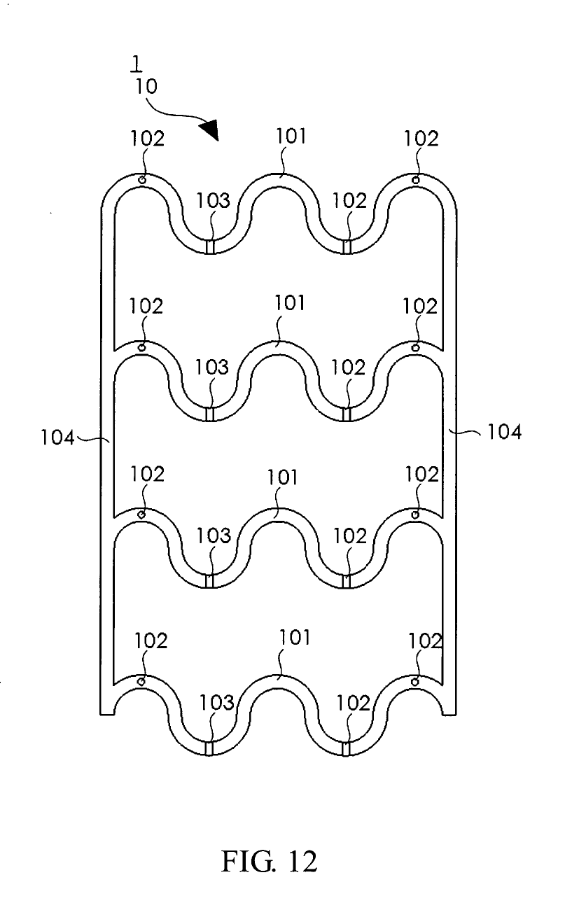

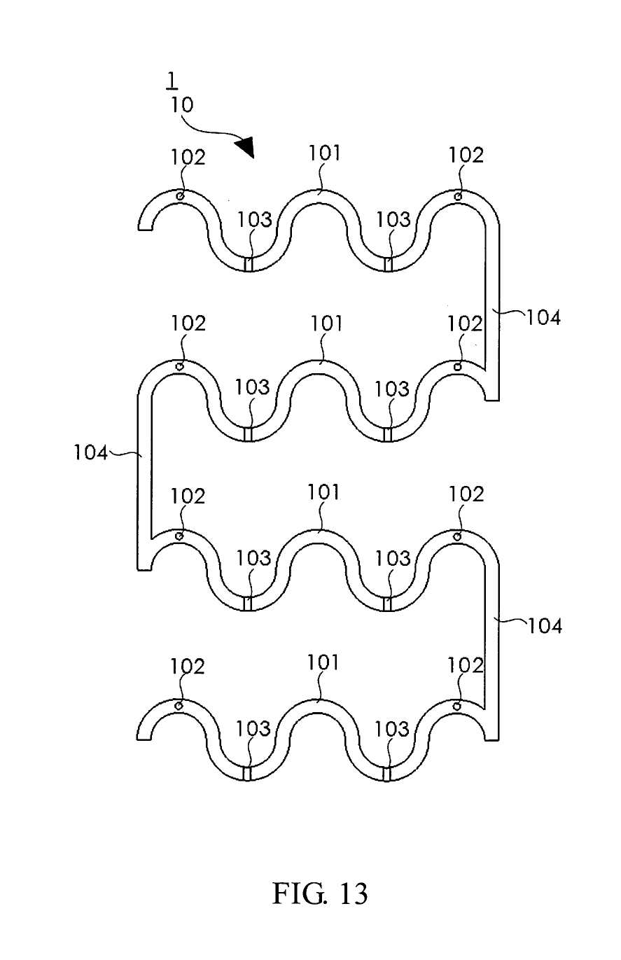

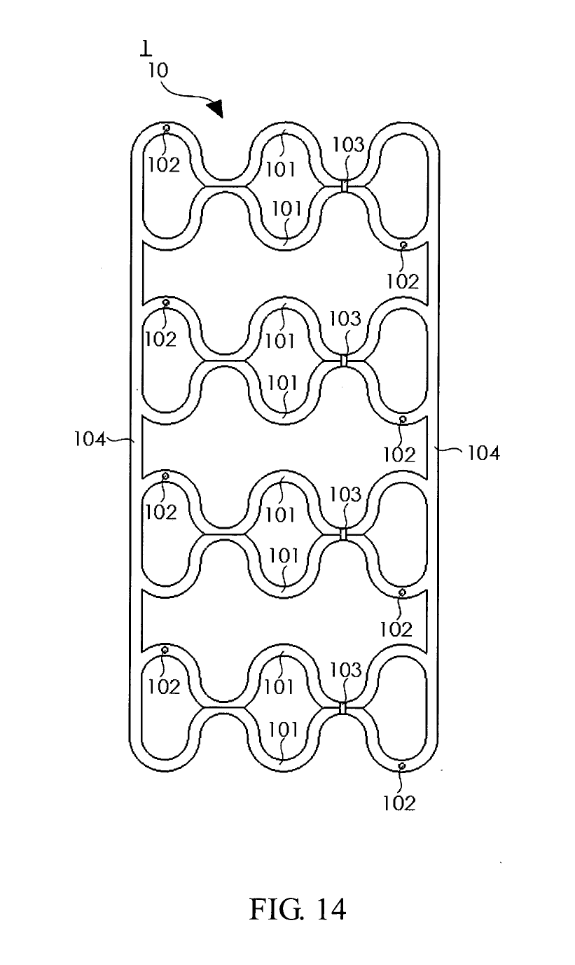

Referring to FIGS. 11˜14 which illustrate different types of struts (101) and supporting struts (104) in the third embodiment and the main body (10) comprising a plurality of struts (101), a plurality of punched holes (102), a plurality of indentations (103) and at least a supporting strut (104) wherein the punched holes (102) and the indentations (103) installed and functional here are similar to those of the first embodiment and not repeatedly interpreted. In this regard, the struts (101) are designed to be patterns not overlapping one another, and the supporting struts (104) are arranged in lateral margins of the main body (10) and used to connect the struts (101). The struts (101) could be developed to be non-staggered patterns (FIG. 12) or staggered patterns (FIG. 14), respectively; the lateral margins of the main body (10) comprise one supporting strut (104) only (FIG. 11) or a plurality of continuous supporting struts (104) (FIG. 12) or discontinuous supporting struts (104) (FIG. 13).

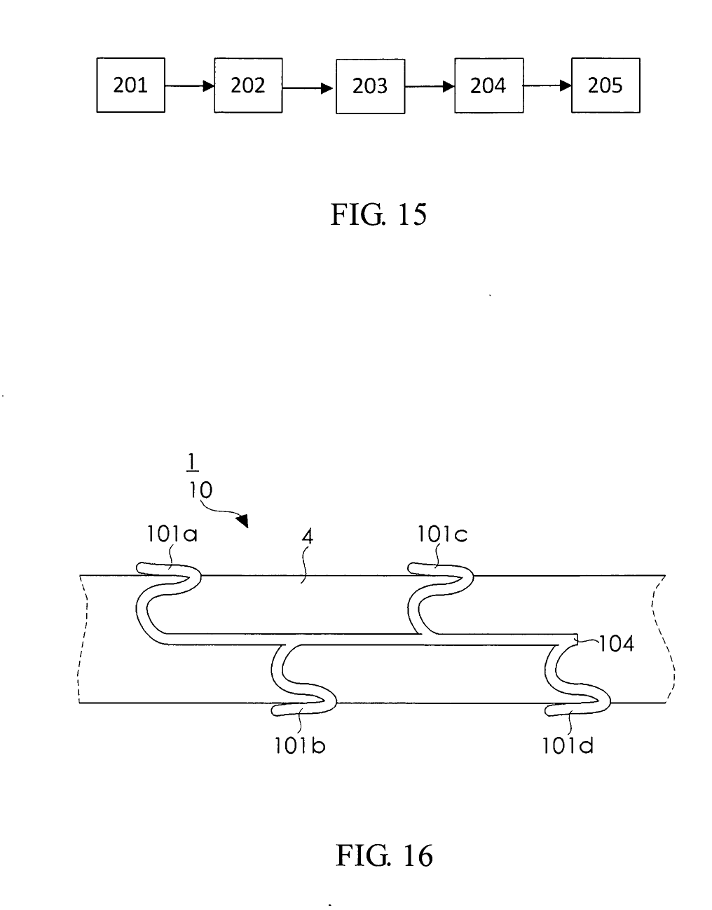

As a result of the struts (101) designed to be non-overlapping patterns in this embodiment, the manufacturing method in the third embodiment (FIG. 15) different from the first one consists in patterns drawn in Step b which comprise one main body only without a rolling & forming step as shown in Step d of the first embodiment but has other manufacturing steps identical to those of the first embodiment.

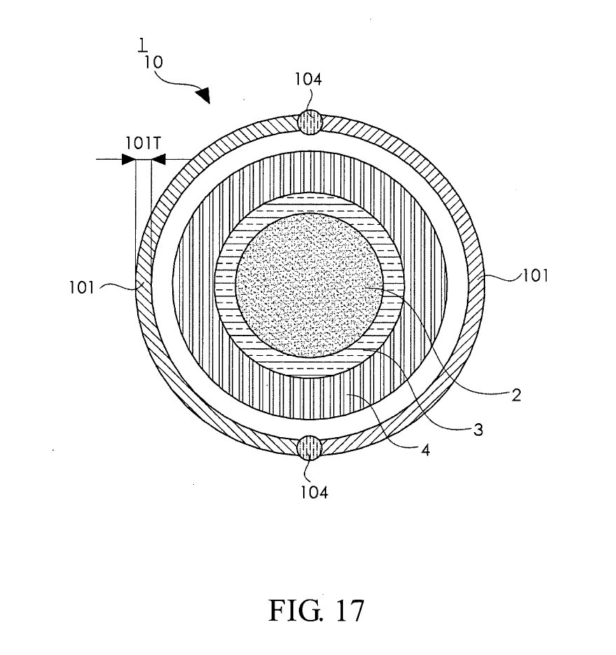

In detail, referring to FIGS. 16 and 17 which illustrate an axle (2) staggering and penetrating among the struts (101a; 101b; 101c; 101d); from positions at the strut (101) and the axle (2) (FIG. 17), the strut (101a or 101c) and the follow-up strut (101b or 101d) (FIG. 16) are located on the left-hand side and the right-hand side of the axle (2), respectively, so that the present invention fabricated without a rolling & forming step is developed to be a tubular stent with the axle (2) staggering and penetrating among the struts (101a; 101b; 101c; 101d) and expands with a dilated balloon up to a required diameter during PTCA.

Certainly, a stent's length (width and external size) and an axle's or a stent's diameter with a balloon dilating during PTCA should depend on an application or a section of one blood vessel for a stent to be implanted but not confine struts' staggered patterns in the present invention, either two struts (each one on the right and left hand sides, respectively) or four struts (every two on the right and left hand sides, respectively) staggering for penetration of the axle.

The stent in the present invention comprising a main body or at least a strut is manufactured with a punching die which is used in precision punching machining without a follow-up process of precision welding. Therefore, the present invention is effective in improved yield rate, mass production, effectively curtailed throughput time and manufacturing cost, and lowered price benefiting more patients.

As a result, the present invention featuring its effects different from general conventional stents as well as manufacturing methods and referred to as creative work among similar products meets patentability and is applied for the patent.

It must be stressed that the said disclosures demonstrate the preferred embodiments of the present invention only and any equivalent change in disclosures, claims or drawings with respect to the present invention is rationally covered by claims of the present invention.

What is claimed is:

1. A punching-based stent used to support a narrowed or occlusive blood vessel comprises:
A stent (1) with a main body (10) and a plurality of bulges (11):
the bulges (11) located at two opposite lateral margins of the main body (10) and arranged in staggered patterns; the main body (10) comprising a plurality of struts (101) and at least a supporting strut (104); the bulge (11) comprising at least an auxiliary strut (111); the supporting strut (104) installed at not less than one lateral margin of the main body (10) and connected to the struts (101) as well as the auxiliary strut (111) of the bulge (11).

2. The punching-based stent according to claim 1 wherein the struts (101) are distributed in staggered or non-staggered patterns.

3. The punching-based stent according to claim 1 wherein the auxiliary strut (111) is distributed in a cannular staggered or non-staggered pattern.

4. The punching-based stent according to claim 1 wherein the strut (101) comprise at least a punched hole (102) or at least an indentation (103); the auxiliary strut (111) comprises at least a punched hole (112) or at least an indentation (113).

5. The punching-based stent according to claim 1 wherein the thickness (101T) of the strut (101) or the thickness (111T) of the auxiliary strut (111) is kept between 0.01 mm and 0.3 mm.

6. The punching-based stent according to claim 1 wherein the stent (1) is manufactured in (composite) metallic materials such as stainless steel, cobalt alloy, nickel, tantalum, and titanium-nickel alloy or polymer materials.

7. A punching-based stent used to support a narrowed or occlusive blood vessel comprises:
A stent (1) comprising at least a strut (12) bended to at least two supporting struts (121a, 121b) in which there is not less than a fold (13) at their intersection.

8. The punching-based stent according to claim 7 wherein the included angle (A) developed by the supporting struts (121a, 121b) is less than 180 degrees.

9. The punching-based stent according to claim 7 wherein the strut (12) comprises at least a punched hole (102) or at least an indentation (103).

10. The punching-based stent according to claim 7 wherein the stent (1) is manufactured in (composite) metallic materials such as stainless steel, cobalt alloy, nickel, tantalum, and titanium-nickel alloy or polymer materials.

11. A punching-based stent used to support a narrowed or occlusive blood vessel comprises:
A stent (1) with a main body (10) comprising a plurality of struts (101) and at least a supporting strut (104); the struts (101) designed to be non-overlapping patterns; the supporting strut (104) located at not less than one lateral margin of the main body (10) and connected to the struts (101).

12. The punching-based stent according to claim 11 wherein the strut (101) comprises at least a punched hole (102) or at least an indentation (103).

13. The punching-based stent according to claim 11 wherein the thickness (101T) of the strut (101) is kept between 0.01 mm and 0.3 mm.

14. A method of manufacturing a punching-based stent comprises steps as follows:
a. Select one material used to manufacture a stent (201);
b. Design and draw a stent's patterns (202);
c. Manufacture a punching die according to the stent's patterns designed and drawn (203);
d. Develop at least one stent Work-in-Process (WIP) from the material by means of the punching die for precision punching machining (204); and
e. Complete the stent with burrs and rough surfaces on the stent removed (206).

15. The method of manufacturing a punching-based stent according to claim 14 wherein the patterns drawn in Step b comprise a main body or a plurality of bulges.

16. The method of manufacturing a punching-based stent according to claim 14 wherein the patterns drawn in Step b comprise at least a strut bended to not less than two supporting struts in which there is at least one fold at these supporting struts' intersection.

17. The method of manufacturing a punching-based stent according to claim 14 wherein there is at least one jig used to clamp or position the material for a precision punching machining process successfully performed by a punching die during Step d.

18. The method of manufacturing a punching-based stent according to claim 14 wherein there is one post-inspection process adopted to check integrity of a stent manufactured in Step d.

19. The method of manufacturing a, punching-based stent according to claim 14 wherein there is one rolling & forming step after Step d by which the stent WIP could be rolled to a cylindrical shape (205).

20. The method of manufacturing a punching-based stent according to claim 14 wherein some substances such as heparin and titanium oxide or sprayed degradable (or non-degradable) polymer film agents could be applied to or coated on the stent's surface.
